(19) United States
(12) Patent Application Publication
(10) Pub. No.: US 2011/0087144 A1
(43) Pub. Date: Apr. 14, 2011

(54) DISPOSABLE SHOWER PROTECTIVE COVER

(76) Inventors: LYNDA LEE, Fredericksburg, VA (US); Meagan Simmons, Rhoadesville, VA (US)

(21) Appl. No.: 12/899,643

(22) Filed: Oct. 7, 2010

Related U.S. Application Data
(60) Provisional application No. 61/250,695, filed on Oct. 12, 2009.

(51) Int. Cl. A61F 5/00

(52) U.S. Cl. 602/3

(57) ABSTRACT

A disposable shower protective cover includes a flexible tubular crisscross patterned sleeve. A plurality of elastic bands are attached to the flexible tubular sleeve at predetermined spaced apart intervals from each other, so as to encircle the flexible tubular sleeve and hold the flexible tubular sleeve in place in a substantially watertight seal against a skin surface of a person when bathing and showering. The sleeve material is transparent so the person or patient can see through for the location of the wounds or IV sites. The cover can be cut in a customized specific length from a spool.

### CROSS-REFERENCE TO RELATED APPLICATION

This application claims the benefit of Provisional Patent Application No. 61/250,695, filed on Oct. 12, 2009, in the United States Patent & Trademark Office, the disclosure of which is incorporated herein by reference.

### BACKGROUND OF THE INVENTION

1. Field of the Invention

The present invention relates to a waterproof shield, and more particularly, a disposable protective cover.

The disposable protective cover allows users to protect the integrity and dryness of an injury or medical sites during normal, everyday activities. The present invention being waterproof can be placed over casts, IV sites, skin irritations and wound dressings to keep these sites dry while bathing or showering. Hospital staff, home health agencies or private individuals can help keep patients clean and sanitary by using the disposable protective cover. The present invention is intended for a one-time use to prevent bacteria growth on the medical site. A flexible tubular sleeve of the disposable protective cover and packaging allows users to roll out the desired amount of the flexible tubular sleeve and easily cut it to size, preventing costly per-unit packaging. A storage box conveniently fits in linen closets or supply room shelves.

2. Description of the Prior Art

There are many people undergoing medical procedures that result in patients needing to wear a plaster cast, IV, or wound dressing. These sites are fragile and need to stay dry in order for proper healing to take place. However, it is also important that the patient maintain high levels of personal hygiene and that they bath or shower frequently to keep from spreading bacteria. These two needs clash to create an inherent problem. Many other products currently available on the market are bulky, must be inflated, and can be time consuming to use. Many are not disposable creating a risk of contamination and infection. A better option is needed.

Numerous innovations for water resistant covers have been provided in the prior art that will be described. Even though these innovations may be suitable for the specific individual purposes to which they address, however, they differ from the present invention.

U.S. Pat. No. 4,911,151, Issued on Mar. 27, 1990, to Rankin et al. teaches a disposable waterproof covering for a cast or bandage on an arm or leg to be used primarily when bathing or showering to protect against wetting the cast or bandage. A generally tubular plastic sleeve is sealed to the user's armor leg above the cast or bandage by a flexible plastic strap to affect a snug, water-tight contact of the sleeve with the arm or leg. The strap is secured by an adhesive at one end to the sleeve adjacent the open end of the sleeve and wound around 360 degrees and secured at its other by a pressure sensitive adhesive.

U.S. Pat. Pub. No. 2005/0027227, Published on Feb. 3, 2005, to Dumas et al. teaches a disposable, water resistant covers having a pair of resilient bands at each open end, and an absorbent medium disposed between the pair of resilient bands. The bands provide a substantially watertight seal between the cover and the user's skin surface. The absorbent medium prevents any moisture that manages to bypass one band to reach the other band. The covers are formed to cover any medical application such as bandages, casts, splints, etc., on a patient's head, neck, chest, waist, arms, legs, and feet to protect against water seepage when attending to regular personal hygiene.

U.S. Pat. Pub. No. 2007/0078364, Published on Apr. 5, 2007, to Davis Belcher teaches a stabilizing cover to wear over an IV site or any type of wound that requires a protective pad and needs to be accessed or observed often. The device's opening/closure allows doctors, nurses, and other care-givers to check the wound site without the discomfort and inconvenience of removing adhesive tape that is typically used to secure a bandage to the wound site. Using the present invention, IV sites may be checked or changed without pulling up or down and possibly pulling the IV out. The cover also can secure gauze or other wound coverings over the wound site without use of adhesives on the patient's skin. Various sizes of snag-free fasteners, in accordance with the present invention, are used to simplify and expedite the process of checking the site. The stabilizing cover is made of fabric that is flexible, comfortable and disposable.

U.S. Pat. No. 7,290,290, Issued on Nov. 6, 2007, to Treadway Fancher teaches a disposable, yet re-usable, clear plastic protective covering, preferably clear, but not limited in color or clarity, designed to enclose articles or areas of the body in an air tight, thus waterproof environment and fitted to the body by means of simple single or multiple bands of elastic, permanently attached to the open end(s) by heat sealing, sewing or gluing with an adhesive, around the circumference of the device opening, to secure the device in place for the duration of use providing single-handed application and removal, and preventing exposure to damaging moisture, fluids or other contaminants to the area of concern for use in medical or non-medical situations.

U.S. Pat. No. 7,662,116, Issued on Feb. 16, 2010, to Ritchey teaches a cover for preventing wetness or contamination of an intravenous site. The cover is manufactured in a variety of sizes and can fit on any extremity of the human body.

U.S. Pat. No. 4,178,924, issued on Dec. 18, 1979, to Samuel H. Baxter describes an elongated tubular protective cover for a cast enclosed body limb portion is provided and at least one end of the protective cover is open and includes an elongated flexible seal band of water impervious material carried by one end of the cover and for wrapping about that end and the adjacent limb portion disposed outwardly of the cover in an edge overlapping spiral manner in order to form a fluid tight seal between the cover and the adjacent limb portion.

U.S. Pat. Pub. No. US2001/0041853 A1, published on Nov. 15, 2001, to Robert W. South et al., describes a cast cover configured to cover a specific portion of a user's body such as a leg, an arm, the torso or head includes an integral, elastomeric band adjacent an open end to provide a water tight seal between the cover and user's body.

U.S. Pat. No. 5,817,038, issued on Oct. 6, 1998, to Beatrice M. Orange et al., describes a system for supporting and protecting wounds, incisions, transdermal procedural sites, and associated medical equipment from moisture and other contaminants.

U.S. Pat. No. 4,639,945, issued on Feb. 3, 1987, to John J. Betz, describes a shield to protect a limb which has been injured, is using an orthopedic device, bandage, or the like, or otherwise requires protection from water during bathing of patients or the like.

U.S. Pat. No. 5,063,919, issued on Nov. 12, 1991, to Doris C. Silverberg, describes a water proof member protects casts, splints, or other appliances or surgical dressings from water damage. A combination of a securing member and straps secure the member and provide a water proof seal between the water proof member and the wearer.

U.S. Pat. No. 5,455,970, issued on Oct. 10, 1995, to Ricardo b. Bance et al., describes an adjustable shower and rain cap is provided, which consists of a headband worn about a head of a person. A waterproof pliable crown is affixed to the headband, so as to extend over the head of the person at different changeable heights.

U.S. Pat. No. 4,562,834, issued on Jan. 7, 1986, to Norman Bates et al., describes a waterproof covering intended to be worn over a cast or bandage on an injured arm or leg. The covering comprises a generally tubular water impervious sleeve which is closed at one end and which has spaced cutting sites running from side to side along which the sleeve can be cut to different lengths.

U.S. Pat. No. 3,329,144, issued on Jul. 4, 1967, to Peter C. Liman, describes a cast protector for arms and legs.

U.S. Pat. No. 3,785,374, issued on Jan. 15, 1974, to Herbert G. Lipson, describes an elongated waterproof, flexible bag having an opening at on end through which a cast-bound limb is inserted and subsequently sealed about the limb with an inflatable cuff.

U.S. Pat. No. 6,191,335, issued on Feb. 20, 2001, to Joseph W. Robinson, describes a wound dressing consisting of a backing layer of hydrophilic polyurethane, an apertured or net wound facing layer of polyurethane, and an intermediate absorbent layer comprising a cotton fiber support layer containing a polyacrylic acid based superabsorber material. The absorbent fiber material will form a gel upon contact with any moisture. The device is distinguishable for requiring cotton fibers and polyacrylic acid based superabsorber material backed by a hydrophilic polyurethane layer.

U.S. Pat. No. 5,715,841, issued on Feb. 10, 1998, to Leo J. Utecht, describes a personal protection apparatus with an adhesive useful for covering burn areas, wound areas and contaminated surfaces of various kinds. The barrier comprises layers of absorbent material that may be a gel, hydrogel, hydrophobic web, or natural or synthetic fibrous material. The devices are distinguishable for requiring various gels, hydrogels, hydrophobic webs, or synthetic fibrous materials.

U.S. Pat. No. 6,512,158 B1, issued on Jan. 28, 2003, to John A. Dobos describes a medical protective wrap device comprising a rectangular liquid impermeable and stretchable polymeric wrap that provides a watertight seal and barrier for wounds and seepage.

U.S. Pat. Pub. No. 2002/0091347 A1, published on Jul. 11, 2002, for Thomas G. Eakin, describes a wound dressing composition comprising an inner covering release layer consisting of paper coated with either polyethylene or silicon, a dressing layer comprising a gelling layer of either carboxymethyl cellulose alone with starch or sticky polyisobutylene (optionally mixed with polyethylene), and an indicator layer comprising dots of a soluble dye mixed with sodium carboxymethyl cellulose or polyisobutylene. Moisture from the wound would cause the activation of the indicator layer. The wound dressing composition is distinguishable for requiring activation of dyes by leaking moisture.

U.S. Pat. Pub. No. 2002/0156411 A1, published on Oct. 24, 2002, for Helge Ahrens et al., describes a dressing composition comprising a top layer of a transparent multilayered polyurethane film over an adhesive layer comprising a polyacrylate including a hydrocarbon tackifier added to enhance adhesion to the skin. The dressing is beveled from its center. The dressing composition is distinguishable for requiring a beveled shape, a multilayered polyurethane film, and an adhesive layer of a polyacrylate and a hydrocarbon tackifier.

U.S. Pat. No. 4,523,586, issued on Jun. 18, 1985, to Mark S. Couri, describes a protective cover for a limb or a cast comprising a flexible fabric bag having hook and loop fastening patches or snaps to close the bag about a limb tightly. The covers are distinguishable for requiring only fabric bags and fastening.

U.S. Pat. No. 5,048,122, issued on Sep. 17, 1991, to Deborah G. Prieur, describes a baby garment or creeper for shielding lines such as catheters to the infant patient comprising a close fitting soft cotton garment body having a zippered front portion. The garment is distinguishable for lacking waterproofing.

U.S. Pat. No. 5,063,919, issued on Nov. 12, 1991, to Doris C. Silverberg, describes a waterproof protective sleeve made of polyethylene-vinyl for protecting casts, splints or other surgical dressings on arms, legs, and individual fingers from water damage comprising a combination of a securing tubular polyethylene-vinyl bag member and numerous encircling straps, a non-skid end strip to secure the bag member with the elastic members, and to provide a waterproof seal between the member and the wearer. The devices are distinguishable for requiring numerous encircling straps, end strips, and fingers.

U.S. Pat. No. 5,152,741, issued on Oct. 6, 1992, to Frank G. Famio, describes a surgical female chest or bra dressing comprising a flexible band formed primarily of a stretchable material with free overlapping ends that engage between the breasts. The band includes differential support structure to provide more support for the sides of the user. The surgical chest dressing is distinguishable for its required bra structure and the lack of a plastic waterproof cover.

U.S. Pat. No. 5,257,956, issued on Nov. 2, 1993, to Carol J. Ewen, describes a post-mastectomy garment comprising a padded vest-like garment adapted for applying comforting pressure to the sites of breast removal and other tissues, and for holding pain relieving packages. The garment is distinguishable for being limited to a vest adapted to hold packages.

U.S. Pat. No. 5,395,302, issued on Mar. 7, 1995, to Rudolph P. Botha et al., describes a protective waterproof sheath for a bandaged injured limb or in a cast comprising a polyvinyl bag made by sealing two sheets to fit a hand and forearm. The sheath can have both ends open to fit an elbow or a knee. The end has a closing strap that is more plasticized than the bag, and attached with hook and loop fastening patches. The device is distinguishable for requiring straps to prevent wetting of the bandaged region and the lack of the inventive gelling seals.

U.S. Pat. No. 5,562,642, issued on Oct. 8, 1996, to James A. Smith et al., describes separately packaged applicator pads for topical delivery of two incompatible dermatological drugs comprising a plurality of rectangular compartmentalized applicator pads that may be exposed and sequentially applied to the afflicted skin area. The device is distinguishable for requiring a plurality of compartmentalized applicator pads.

U.S. Pat. No. 6,503,526 B1, issued on Jan. 7, 2003, to Duane G. Krzysik et al., describes absorbent article compositions containing natural fats, natural oils, sterols or sterol derivatives, emollients, solidifying agents, and viscosity enhancers. The absorbent article comprises an outer cover, a liquid permeable bodyside liner, an absorbent body, and the chemical composition. The articles are distinguishable for requiring the addition of a specific dissimilar chemical composition.

U.S. Pat. No. 6,511,467 B1, issued on Jan. 28, 2003, to Denise Reuss, describes a fabric drainage reservoir medical garment including one or more drainage reservoirs in pockets. The medical garments are distinguishable for requiring pockets to hold drainage reservoirs.

European Patent Application No. EP 1 184 039 A2, published on Mar. 6, 2002, for Yoshihide Murakami et al., describes an adhesive tape or sheet for application skin made of an acrylic copolymer having a specific carboxylic acid ester, wherein the acrylic copolymer has a gel fraction adjusted to a specific range. The adhesive tape or sheet can be used for first aid adhesive plasters, surgical tapes, large adhesive plasters with a pad, dressing materials, and the like. The device is distinguishable for being limited to an adhesive tape or sheet requiring acrylic copolymers.

It is apparent now that numerous innovations for water resistant covers have been provided in the prior art that are adequate for various purposes. Furthermore, even though these innovations may be suitable for the specific individual purposes to which they address, accordingly, they would not be suitable for the purposes of the present invention as heretofore described.

### SUMMARY OF THE INVENTION

AN OBJECT of the present invention is to provide a disposable protective cover that avoids the disadvantages of the prior art.

ANOTHER OBJECT of the present invention is to provide a disposable protective cover that is simple and inexpensive to manufacture.

STILL ANOTHER OBJECT of the present invention is to provide a disposable protective cover that is simple to use.

BRIEFLY STATED, STILL YET ANOTHER OBJECT of the present invention is to provide a disposable protective cover which comprises a flexible tubular sleeve. A plurality of elastic bands are attached to the flexible tubular sleeve at predetermined spaced apart intervals from each other, so as to encircle the flexible tubular sleeve and hold the flexible tubular sleeve in place in a substantially watertight seal against a skin surface of a person or patient when bathing and showering.

The novel features which are considered characteristic of the present invention are set forth in the appended claims. The invention itself, however, both as to its construction and its method of operation, together with additional objects and advantages thereof, will be best understood from the following description of the specific embodiments when read and understood in connection with the accompanying drawing.

### BRIEF DESCRIPTION OF THE DRAWING

The figures of the drawings are briefly described as follows:

FIG. 1 is a perspective view showing two configurations of the present invention;

FIG. 2 is a perspective view showing an arm of a person or patient being inserted into the present invention;

FIG. 3 is a perspective view showing the present invention in use;

FIG. 4 is a perspective view showing the present invention being dispensed from a storage box; and

FIG. 5 is a top view of the storage box with the cover removed, showing the present invention rolled up therein.

### REFERENCE NUMERALS UTILIZED IN THE DRAWING

110 disposable protective cover
112 flexible tubular sleeve of cover 110
114 elastic band of cover 110
116 skin surface of person or patient 118
118 person or patient
120 thin flexible waterproof plastic material for flexible tubular sleeve 112
122 lightweight elastic patterns in flexible tubular sleeve 112
124 print on flexible tubular sleeve 112
126 arm of person or patient 118
128 IV site on arm 126
130 storage box
132 spool in storage box 130
134 pair of scissors

### DETAILED DESCRIPTION OF THE PREFERRED EMBODIMENT

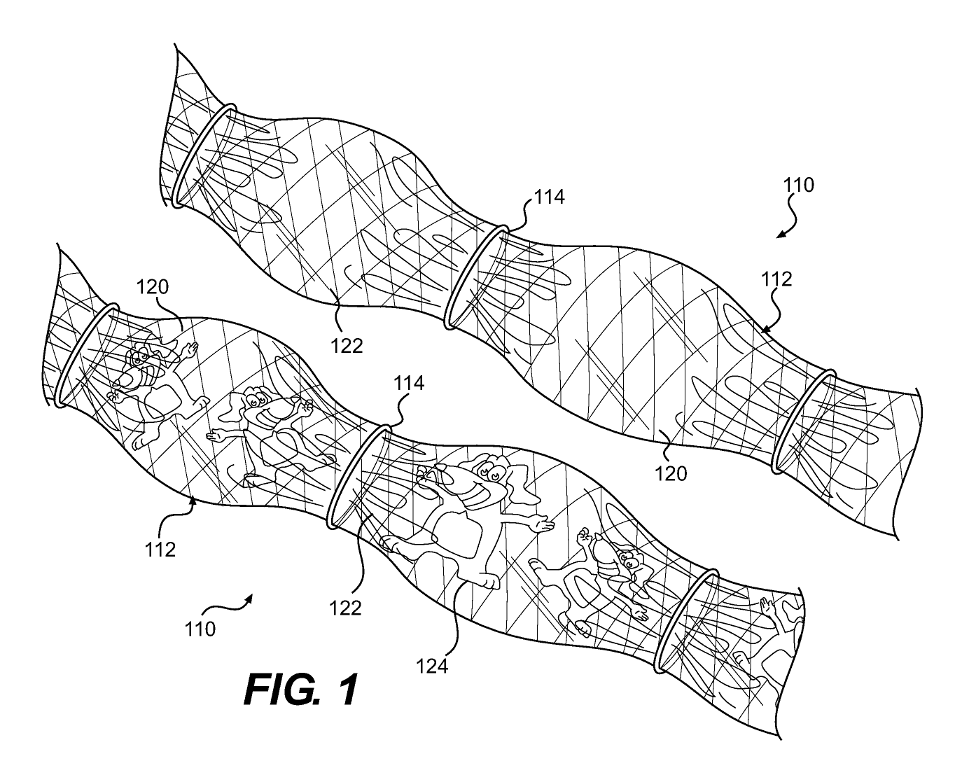

Referring now to the figures, in which like numerals indicate like parts, and particularly to FIGS. 1 through 5, and as such, will be discussed with reference thereto.

The present invention is a disposable protective cover 110 which comprises a flexible tubular sleeve 112. A plurality of elastic bands 114 are attached to the flexible tubular sleeve 112 at predetermined spaced apart intervals (e.g. 3 inches, 4 inches, . . . etc. . . . ) from each other, so as to encircle the flexible tubular sleeve 112 and hold the flexible tubular sleeve 112 in place in a substantially watertight seal against a skin surface 116 of a person or patient 118 when bathing and showering. For example, the elastic bands can form a circle with a diameter of minimum 1.5 inches (or minimum length of 3 inches) and expand to a diameter of 4 inches (or minimum length of 8 inches).

The flexible tubular sleeve 112 is comprised out of a thin flexible waterproof plastic material 120. The material 120 is also transparent so the patient can see through for the location of the wounds or IV sites. The material 120 can be polyethylene (PE), polyethylene vinyl acetate (PEVA), Chlorinated polyethylene (CPE), polyvinyl chloride (PVC), and polypropylene (PP) plastic. The sleeve 112 has a thickness of 0.01 mm-0.05 mm. At least, a lightweight elastic pattern 122 is crisscrossed and thermally embedded or heat fusingly incorporated into the flexible tubular sleeve 112. The lightweight elastic patterns 122 are multithreads elastic bands forming in a mesh or matrix. The elastic bands 114 are spaced apart on the flexible tubular sleeve 112 at approximately three to four inches. The elastic band 114 can be vulcanized rubber, polyethylene (PE), polyethylene vinyl acetate (PEVA), Chlorinated polyethylene (CPE), polyvinyl chloride (PVC), or polypropylene (PP) plastic. The elastic band 114 has a diameter (0.02 mm-0.1 mm) twice the thickness of the sleeve 112. The lightweight elastic pattern 122 can be fiber, vulcanized rubber, polyethylene (PE), polyethylene vinyl acetate (PEVA), Chlorinated polyethylene (CPE), polyvinyl chloride (PVC), or polypropylene (PP) plastic. The lightweight elastic pattern 122 can have same thickness of the sleeve 112.

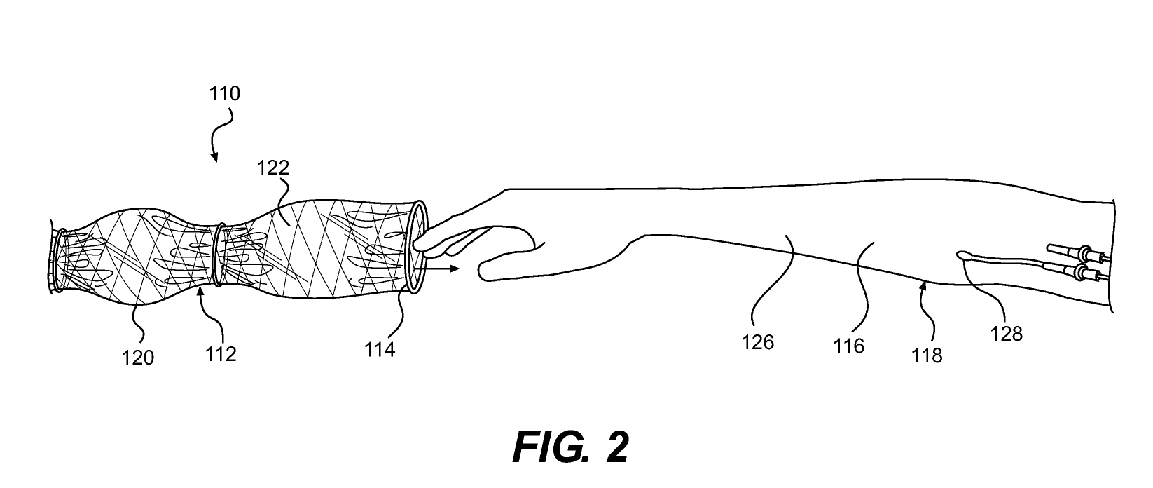

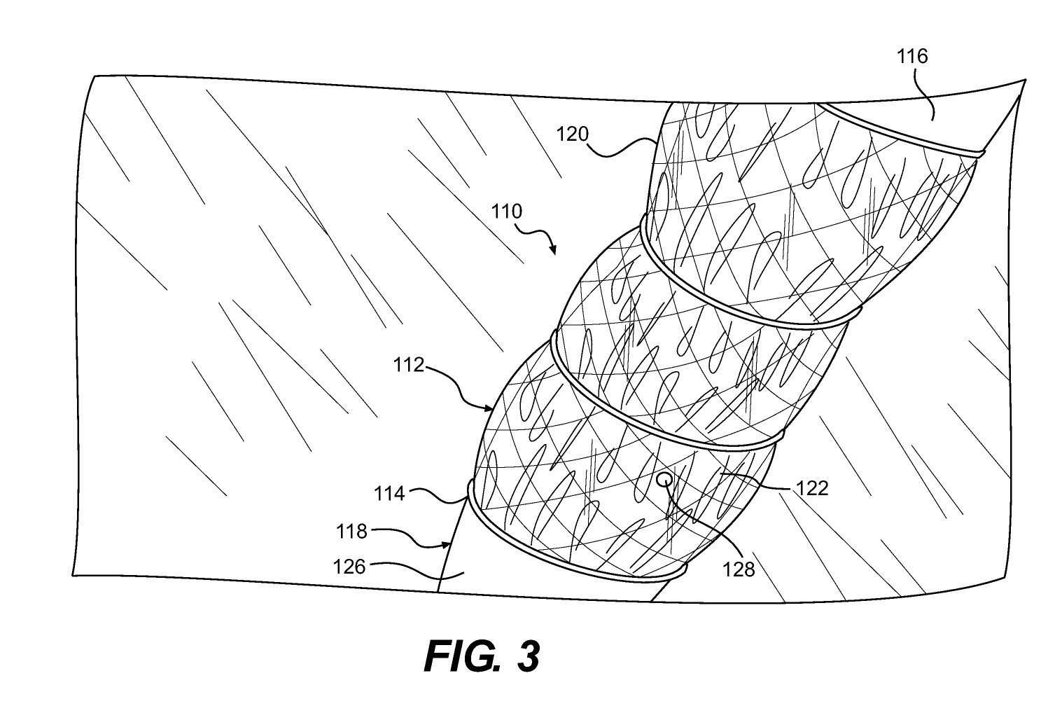

As best seen in FIG. 1, the configuration of the flexible tubular sleeve 112 is selected from the group consisting of plain, colors and prints 124, such as cartoon characters, animals and sports logos. The flexible tubular sleeve 112 is adapted to protect a body portion of the person or patient 118 selected from the group consisting of an arm 126 (as seen in FIGS. 2 and 3), a leg and a torso (not shown).

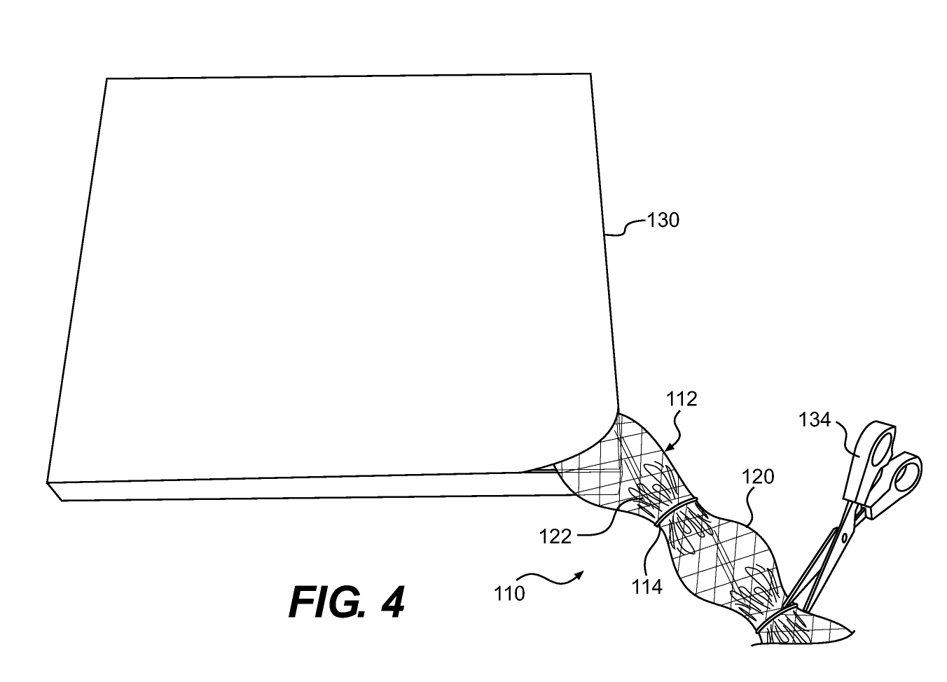

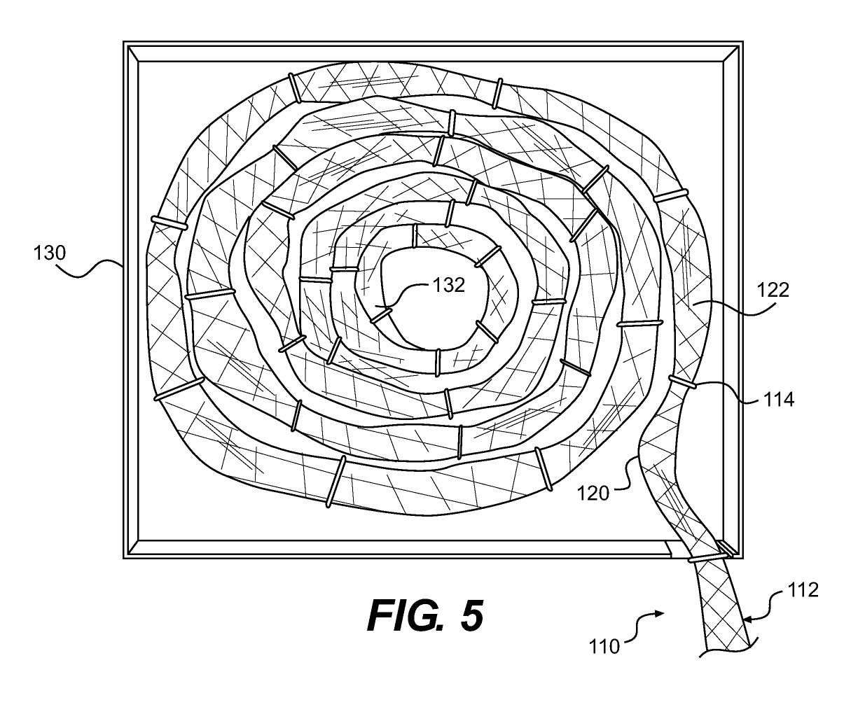

The flexible tubular sleeve 112 is adapted to protect an area on a body portion of the person or patient 118 selected from the group consisting of an IV site 128, a plastic cast, a skin irritation and a wound dressing (not shown). As shown in FIGS. 4-5, a storage box 130 is provided. A spool 132 is carried within the storage box 130, with the flexible tubular sleeve 112 configured in an elongated strip and rolled upon the spool 132. The person or patient 118 can dispense a customized specific length of the flexible tubular sleeve 112 from the storage box 130 by cutting it off with a pair of scissors 134.

In summary the disposable protective cover 110 helps patients keep the IV sites 128 and plaster casts dry and in good condition. The flexible tubular sleeve 112 is made out of the thin flexible waterproof plastic material 120. The material 120 is also transparent so the patient can see through for the location of the wounds or IV sites. The material 120 is similar to the type used in disposable shower caps or other suitable waterproof items. The flexible tubular sleeve 112 is intertwined with the elastic bands 114 to hold it in place. The elastic bands 114 occur at every three to four inches. The lightweight elastic pattern 122 is crisscrossed and heat fusingly incorporated into the flexible tubular sleeve 112 to help keep the flexible tubular sleeve 112 snug against the skin surface 116. The flexible tubular sleeve 112 is packaged with the spool 132 within the storage box 130, allowing the person or patient 118 to easily dispense the amount of the flexible tubular sleeve 112 they need. The person or patient 118 will then take the pair of scissors 134 to cut the flexible tubular sleeve 112 in between the elastic bands 114. The flexible tubular sleeve 112 may be available in plain, colors, or prints 124 such as cartoon characters, animals, business names or sports logos. The flexible tubular sleeve 112 may be available in various sizes for the person or patient 118, such as an adult or child, on the arm 126, the legs, or the torso. Specifications may vary upon manufacturing.

It will be understood that each of the elements described above, or two or more together, may also find a useful application in other types of constructions differing from the types described above.

While the invention has been illustrated and described as embodiments of a disposable protective cover, accordingly it is not limited to the details shown, since it will be understood that various omissions, modifications, substitutions and changes in the forms and details of the device illustrated and its operation can be made by those skilled in the art without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute characteristics of the generic or specific aspects of this invention.

The invention claimed is:

1. A disposable protective cover which comprises:
a) a flexible tubular sleeve in a spool form,
b) a plurality of elastic bands attached to the flexible tubular sleeve at predetermined spaced apart intervals from each other, so as to encircled the flexible tubular sleeve and hold the flexible tubular sleeve in place in a substantially watertight seal against a skin surface of a person when bathing and showering,
c) lightweight elastic patterns which are crisscrossed and heat fusingly incorporated into the flexible tubular sleeve, and
d) wherein the predetermined spaced apart intervals are at least 3 inches.

2. The cover as recited in claim 1, wherein the flexible tubular sleeve is comprised out of a thin flexible waterproof plastic material and the plastic material is also transparent so the person can see through for the location of the wounds or IV sites.

3. The cover as recited in claim 1, wherein the lightweight elastic patterns are multithreaded elastic bands.

4. The cover as recited in claim 1, wherein the sleeve has a thickness of 0.01 mm-0.05 mm.

5. The cover as recited in claim 1, wherein the sleeve is select from a group consisting of polyethylene (PE), polyethylene vinyl acetate (PEVA), Chlorinated polyethylene (CPE), polyvinyl chloride (PVC), and polypropylene (PP) plastic.

6. The cover as recited in claim 1, wherein the lightweight elastic patterns are select from a group consisting of fiber, vulcanized rubber, polyethylene (PE), polyethylene vinyl acetate (PEVA), Chlorinated polyethylene (CPE), polyvinyl chloride (PVC), or polypropylene (PP) plastic.

7. The cover as recited in claim 1, wherein the lightweight elastic patterns have a thickness of 0.01 mm-0.05 mm.

8. The cover as recited in claim 1, wherein the elastic bands are spaced apart on the flexible tubular sleeve at approximately three to four inches.

9. The cover as recited in claim 1, wherein the configuration of the flexible tubular sleeve is selected from the group consisting of plain, colors, cartoon characters, animals, business names and sports logos.

10. The cover as recited in claim 1, wherein the flexible tubular sleeve is adapted to protect a body portion of the person selected from the group consisting of an arm, a leg and a torso.

11. The cover as recited in claim 1, wherein the flexible tubular sleeve is adapted to protect an area on a body portion of the person selected from the group consisting of an IV site, a plastic cast, a skin irritation and a wound dressing.

12. The cover as recited in claim 1, further comprising:
a) a storage box;
b) a spool carried within the storage box; and
c) the flexible tubular sleeve being configured in an elongated strip and rolled upon the spool, wherein a person can dispense a customized specific length of the flexible tubular sleeve from the storage box by cutting the flexible tubular sleeve off with a pair of scissors.

13. The cover as recited in claim 1, wherein the elastic bands have a thickness of 0.02 mm to 0.1 mm.
